(19) United States
(12) Patent Application Publication
(10) Pub. No.: US 2011/0275895 A1
(43) Pub. Date: Nov. 10, 2011

(54) ENDOTRACHEAL TUBE WITH CAMERA AND ILLUMINATOR AT DISTAL END

(76) Inventor: Robert A. MacKin, Flagstaff, AZ (US)

(21) Appl. No.: 13/107,080

(22) Filed: May 13, 2011

Related U.S. Application Data
(63) Continuation-in-part of application No. 11/865,256, filed on Oct. 1, 2007, now Pat. No. 7,942,813.
Application No. 11/865,256 is a division of application No. 10/775,904, filed on Feb. 10, 2004, now Pat. No. 7,297,105.

(51) Int. Cl. A61B 1/05; A61B 1/267; A61B 1/06

(52) U.S. Cl. 600/112

(57) ABSTRACT

A low cost camera and light emitting diode are coupled to the distal end of an endotracheal tube to obtain an image in real time of tissue at the distal end of the endotracheal tube. Power for the light emitting diode and camera, along with a signal reflective of the image captured by the camera, are transmitted through electrical conductors embedded in the wall of the endotracheal tube and are coupled with a plug. The plug is detachably attached to a radio frequency transmitter module to provide electrical power to the light emitting diode and to the camera and for transmitting the signal of the captured image to a receiver for real time display of the image on a video monitor.

### CROSS-REFERENCE TO RELATED APPLICATIONS

This application is a continuation-in-part of a patent application entitled “Detachable Endotracheal Camera” filed Oct. 1, 2007 and assigned Ser. No. 11/865,256, now U.S. Pat. No. 7,942,813, which is a divisional of a patent application entitled “Endotracheal Camera”, filed Feb. 10, 2004 and assigned Ser. No. 10/775,904, now U.S. Pat. No. 7,297,105, and describing an invention by the present inventor.

### BACKGROUND OF THE INVENTION

1. Field of the Invention

The present invention relates to endotracheal tubes and, more particularly, to an endotracheal tube having an illuminator and a camera at its distal end coupled with electrical conductors to an electrical plug mating with a transmitter to transmit an image from the camera to a receiver for display on a video monitor.

2. Description of the Prior Art

The basic tenets attendant endotracheal tubes having an illuminator at the distal end are illustrated and described in U.S. Pat. No. 5,285,778 and relating to an invention by the present inventor; which patent is incorporated herein by reference. The endotracheal tube described therein includes an optical fiber extending through the endotracheal tube to a viewing lens at the distal end of the tube. An eye piece is attached to the proximal end of the optical fiber to permit viewing through the lens. Illumination of the area under inspection is provided by a high intensity light source extending via the endotracheal tube to an illumination port at the distal end.

### SUMMARY OF THE INVENTION

A light emitting diode is lodged at the distal end of an endotracheal tube to provide illumination of the area of inspection. A camera is also lodged at the distal end of the endotracheal tube to provide an image of the illuminated area. Two pairs of electrical conductors interconnect the diode and the camera with a plug(s) mating with a transmitter module that provides electrical power to the diode and the camera and receives a signal from the camera reflective of the image captured. The transmitter module transmits the signal reflective of the image recorded by the camera to a receiver for displaying the image on a video monitor for viewing.

It is therefore a primary object of the present invention to provide a method for imaging with a camera tissue at the distal end of an endotracheal tube and viewing on a real time monitor the image captured by the camera.

Another object of the present invention is to provide a camera and a source of light at the distal end of an endotracheal tube for obtaining an image captured by the camera.

A yet further object of the present invention is to provide a low power transmitter coupled with a camera located at the distal end of an endotracheal tube to transmit a captured image to a receiver for viewing the image on a video monitor.

Still another object of the present invention is to provide a light emitting diode and a camera at the distal end of an endotracheal tube coupled with electrical conductors to transmit an image to a video monitor for real time viewing.

A further object of the present invention is to provide a small sized inexpensive camera and light emitting diode at the distal end of an endotracheal tube to transmit to a receiver a captured image.

A yet further object of the present invention is to provide a wireless transmission to a video monitor of an image captured by a camera located at the distal end of an endotracheal tube to record the image at the distal end of the endotracheal tube using a low power radio frequency transmitter and receiver.

A still further object of the present invention is to provide a method for displaying an image real time on a video monitor by capturing the image to be displayed with a camera located at the distal end of an endotracheal tube along with a light emitting diode to illuminate the area to be imaged and transmitting the image by a radio frequency transmitter to a corresponding receiver to produce a signal of the image for the video monitor.

A still further object of the present invention is to provide a method for viewing on a video monitor in real time an image at the distal end of an endotracheal tube using a camera located at the distal end of the endotracheal tube and a wireless transmitter and receiver.

These and other objects of the present invention will become apparent to those skilled in the art as the description thereof proceeds.

### BRIEF DESCRIPTION OF THE DRAWINGS

The present invention will be described with greater specificity and clarity with reference to the following drawings, in which:

FIG. 1 is a partial cross-sectional view of an endotracheal tube embodying fiber optics for transmitting an image;

FIG. 2 is a partial cross-section of the endotracheal tube;

FIG. 3 is a partial cross-sectional view illustrating placement within a patient of an endotracheal tube;

FIG. 4 illustrates a camera and a transmitter for attachment with a connector of an endotracheal tube;

FIG. 5 illustrates a receiver and an attached video monitor;

FIG. 6 illustrates an endotracheal tube having a camera and a light emitting diode located at the distal end;

FIG. 7 illustrates a representative cross-section of the endotracheal tube shown in FIG. 6; and

FIG. 8 illustrates placement within a patient of the endotracheal tube shown in FIG. 6.

### DESCRIPTION OF THE PREFERRED EMBODIMENT

Referring to FIG. 1, there is shown an endotracheal tube 10 having a connector 12 for connection to a conventional ventilator to assist a patient's breathing function. The endotracheal tube includes an inflatable balloon 14 in proximity to its distal end 16. The inflatable balloon is inflated by a tube 18 connected through a connector 20 to a small syringe-like air pump after the endotracheal tube has been inserted into a patient's trachea.

Prior endotracheal tubes do not permit any visualization of a patient's tracheal and bronchial passages. If such visualization is needed, connector 12 is disconnected from the ventilator and a conventional bronchoscope is inserted down through hollow passage 21 of the endotracheal tube to allow a physician to determine if a lot of mucus is present in either lung or in either of the left or right stem main bronchi. If it is necessary to suction mucus out of either of the patient's lungs, a suctioning tube is inserted through hollow passage 21. The endotracheal tube may have to be disconnected from the ventilator to allow visualization in the trachea of the lungs or to allow suctioning of the mucus, blood, etc., if the endotracheal tube does not have a sealable side port through which the suctioning tube can be inserted.

When a skilled physician, often a pulmonologist, inserts an endotracheal tube into a patient, it would be desirable for a nurse to be able to easily monitor the position of the endotracheal tube in a patient's trachea to determine if its location has been shifted. If so, the nurse would know whether to call a physician to reposition the endotracheal tube. It would also be desirable to determine accurately the position of the endotracheal tube without requiring an x-ray of the patient.

Still referring to FIG. 1, endotracheal tube 10 includes an optical fiber, hereinafter referred to as fiber optic bundle 22, that extends through the endotracheal tube to a viewing lens 24 at distal end 16. The fiber optic bundle can be an inexpensive plastic optical fiber costing only a few dollars and embedded in the wall of the endotracheal tube. The fiber optic bundle is operatively connected to a connector 26 which includes two prongs 28, 30 of which prong 28 carries the fiber optic bundle. A second plastic optical fiber, hereinafter referred to as fiber optic bundle 32, extends through wall 34 of endotracheal tube 10 to an illumination port 36 at distal end 16.

FIG. 2 is a view of the distal end of endotracheal tube 10. A hollow tube 38 extends from a flushing inlet port connector 40 (see FIG. 1) and extends through the endotracheal tube so that a transparent saline flushing liquid can be forced through the tube to wash mucus away from viewing lens 24 and illumination port 36. Such mucus may collect thereon during insertion of the endotracheal tube into the patient's trachea or afterward.

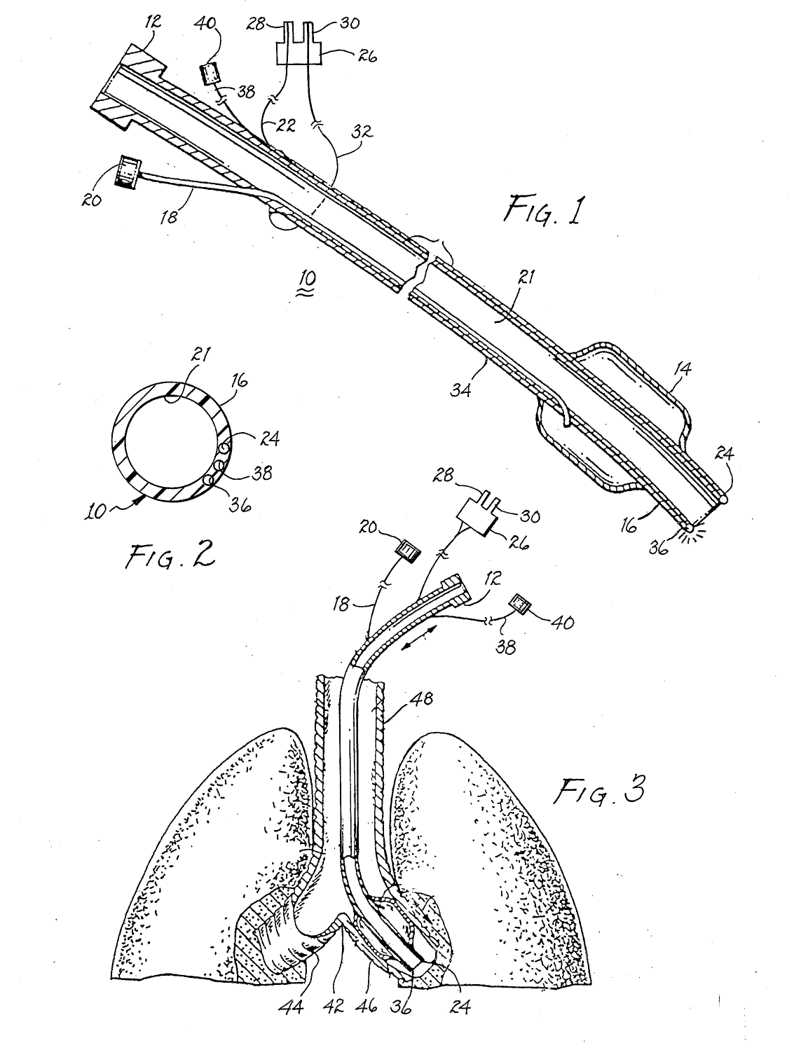

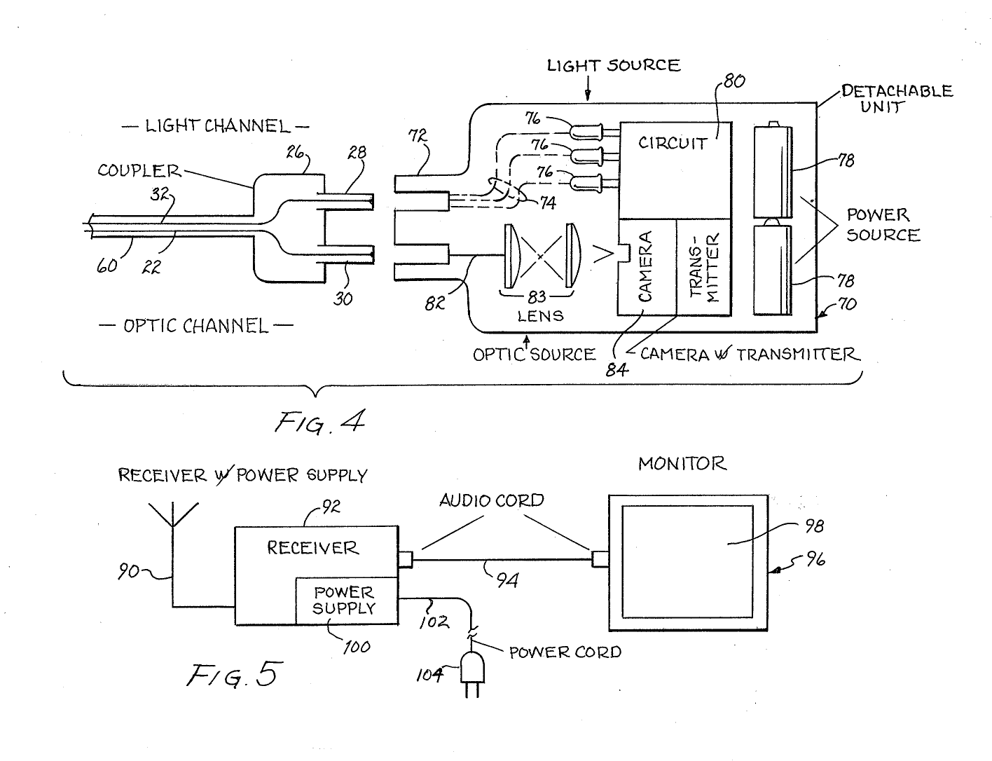

One major advantage of endotracheal tube 10 is that the carina (a cartilaginous structure) 42 (see FIG. 3) can be easily viewed during insertion of the endotracheal tube so that a nurse or a physician can readily determine how far into the patient's trachea to properly insert the endotracheal tube. This avoids the need for an x-ray process to determine if the endotracheal tube is properly inserted. As the endotracheal tube can become malpositioned in the patient and which would normally require a later x-ray to check for proper placement, direct visualization afforded by the present invention can avoid the need for such a repeat x-ray. Another advantage is that the nurse or physician can easily view the conditions in branches 44, 46 of trachea 48 to determine the presence of mucus or other condition and to determine whether there is a need for immediate suctioning of mucus, blood, etc., from either lung or the passages thereto.

Referring to FIG. 4, there is shown a male connector 26 having prongs 28, 30 extending therefrom. Fiber optic bundle 32 is in functional and operative engagement with prong 28 to transmit light from the end of the prong to illumination port 36 at distal end 16 of the endotracheal tube. Fiber optic bundle 22 is coupled with lens 24 at the distal end of the endotracheal tube to transmit light, that is an image, to the end of prong 30. As illustrated, fiber optic bundles 22 and 32 may be incased within a sheath 60.

A removable module 70 includes a female connector 72 for receiving prongs 28, 30 of connector 26. Upon mating of connectors 26, 72, fiber optic bundle 32 within prong 28 is placed in communication with fiber optic bundle 74, the latter being in communication with and receiving light from light emitting diodes 76. Electrical power for the light emitting diodes is provided by circuit 80 connected to batteries 78. Prong 30 of male connector 26 mates with female connector 72 to transmit light, that is, the image visible through lens 24 (see endotracheal tube 10) to convey the received light through a further fiber optic bundle 82 to a lens system 83. The lens system is interconnected with a small sized and relatively inexpensive electronic camera 84. Cameras suitable for this purpose cost less than $100.00 and can be found for less than $50.00 from commercial outlets. The camera is interconnected with a low power radio frequency transmitter 86 to transmit the images recorded by the camera. Transmitters of this type are readily available for less than $100.00 and may be found for less than $50.00 from commercial outlets.

As shown in FIG. 5, an antenna 90 is connected to a radio frequency receiver 92 and receives the images detected by camera 84 and transmitted by transmitter 86. The received image is conveyed via an electrical conductor or cord 94 to a video monitor 96. The video monitor includes a screen 98 for displaying the image recorded by camera 84. As illustrated, a power supply 100 provides power to receiver 92 and to video monitor 96 through an electrical conductor. Power to the power supply may be provided by an electrical conductor connected to a conventional plug 104 for engagement with a conventional wall socket.

In summary, the image conveyed from the lens at the distal end of the endotracheal tube is digitized and recorded by a camera. The image recorded by the camera is displayed real time on a video monitor through a wireless interconnection. The ease of a wireless transmission system in the confines of an operatory avoids the likelihood of a patient and attending health care providers from becoming entangled with cords and wires.

Moreover, presently used wires and cables extending to a video monitor creates a hazard of an attending health care provider inadvertently interfering with such wires and/or cables and causing repositioning or pulling out of the endotracheal tube. This hazard is completely avoided by the present invention due to the absence of such wires and/or cables.

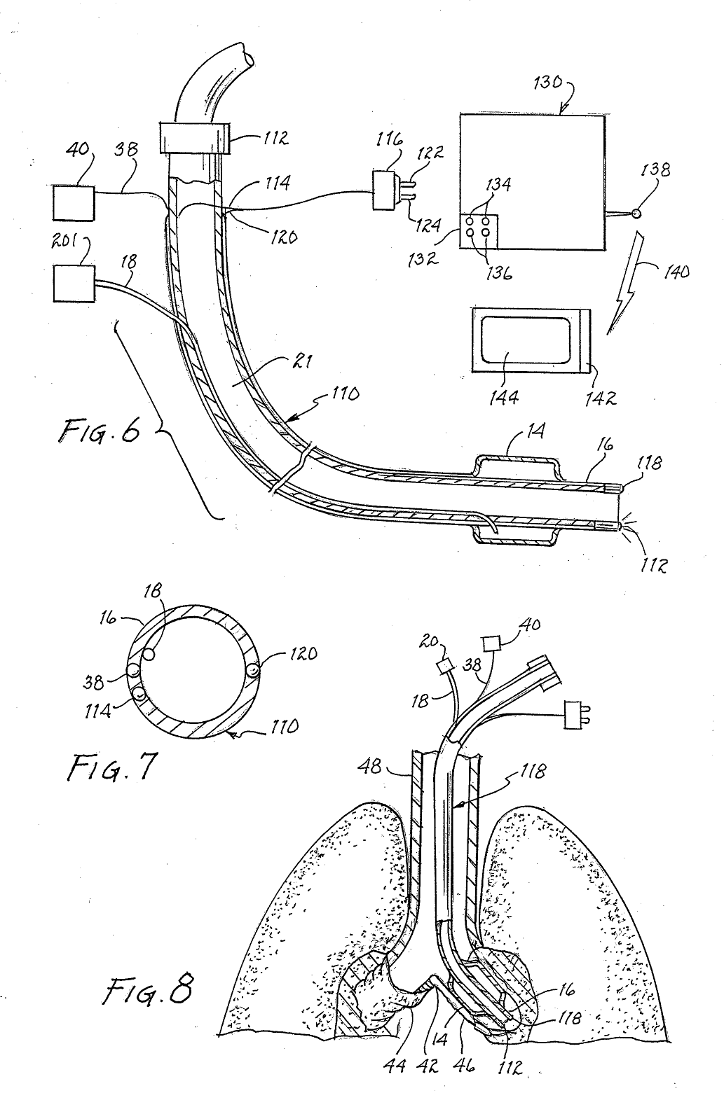

Referring to FIG. 6, there is illustrated a variant of the previously discussed endotracheal tube and identified by numeral 110. The endotracheal tube may be rigid or flexible, depending upon the procedure to be performed. Elements common with endotracheal tube 10 (FIG. 1) have been assigned common reference numerals. To reduce the costs and facilitate operation of endotracheal tube 110, a light emitting diode 112 is disposed at distal end 16. Electrical power for the light emitting diode is provided by an electrical conductor 114. As illustrated in FIG. 7, electrical conductor 114 may be embodied within endotracheal tube 110. A plug 116 is electrically connected to electrical conductor 114 and provides electrical power to the light emitting diode. A miniature camera 118 is also located at distal end 16 of the endotracheal tube. Such miniature cameras are readily commercially available of a size of about one to two millimeters in cross-section. Electrical power for the camera, as well as the signal reflective of the image captured are conveyed through electrical conductor 120. These electrical conductors (114, 120) may be secured to plug 116. Necessarily, each set of electrical conductors attached to the plug is electrically connected to respective prongs 122, 124 extending from the plug.

Electrical conductors 114 may be embedded in the wall of the endotracheal tube, as illustrated in FIG. 7. Similarly, electrical conductors 120 may be embedded within the wall of the endotracheal tube. Alternatively, these electrical conductors may be adjacent the interior or exterior wall of the endotracheal tube.

A low power short range transmitter 130 includes a receptacle 132 in the form of a female plug mating with male plug 116. For example, prongs 122 may mate with receptacles 134 and prongs 124 may mate with receptacles 136. Thereby, power is applied to light emitting diode 112 and camera 118. Furthermore, a signal from the camera reflective of the image captured is conveyed to transmitter 130 via electrical conductors 120. The transmitter includes the necessary electronic/electrical components to provide not only the power required for the light emitting diode and the camera but also processing circuitry for transmitting the signal received from the camera and reflective of the image captured via antenna 138.

The transmitted signal, represented by arrow 140 is received by a receiver 142. The receiver processes the received signal to provide a display on video monitor 144. Thereby, the image captured by camera 118 may be displayed real time on the video monitor to provide a physician or other medical personnel an image of the surface under inspection.

FIG. 8 is similar to FIG. 3 and illustrates the location and function of endotracheal tube 110 when used in a medical procedure. By inspection, it is evident that light emitting diode 112 will illuminate an area/tissue under inspection and such area/tissue will be imaged by camera 118. The resulting image, on a real time basis, will be displayed on video monitor 144. The benefits therefrom are multiple. It provides medical personnel with an image of the area under inspection to permit a determination of a medical procedure necessary or to relocate the endotracheal tube. It ensures that distal end 16 of the endotracheal tube is located in proximity to the tissue to be under inspection; it permits relocation of the distal end as necessary to ensure that the distal end of the endotracheal tube is proximate the area of interest.

Although the use of a light emitting diode to provide illumination may be preferred, lighting the area of interest could also be provided by one or more fiber optic cables, as described above with respect to FIGS. 1, 2 and 4.

I Claim:

1. Apparatus for displaying an image of tissue at the distal end of an endotracheal tube, said apparatus comprising:
(a) a source of light disposed at the distal end of said endotracheal tube for illuminating the tissue to be imaged, said source of light comprising at least one light emitting diode;
(b) electrical conductors extending from said at least one light emitting diode via said endotracheal tube to a location external to said endotracheal tube to provide electric power to said at least one light emitting diode;
(c) a camera disposed at the distal end of said endotracheal tube to capture an image of the area illuminated by said at least one light emitting diode;
(d) further electrical conductors extending from said camera via said endotracheal tube to a location external to said endotracheal tube to provide electric power to said camera and to convey a signal reflective of the image captured by said camera;
(e) a transmitter electrically connected to said electrical conductors and said further electrical conductors for providing electrical power to said at least one light emitting diode and to said camera and for transmitting the signal reflective of the image captured by said camera;
(f) a receiver for receiving the signal transmitted by said transmitter; and
(g) a video monitor coupled with said receiver for displaying the image corresponding with the signal received by said receiver.

2. The apparatus as set forth in claim 1 including a plug electrically connected to said electrical conductors and said further electrical conductors.

3. The apparatus as set forth in claim 2 including a receptacle disposed in said transmitter for receiving said plug.

4. Apparatus for displaying an image of tissue at the distal end of an endotracheal tube, said apparatus comprising:
(a) a source of light disposed at the distal end of the endotracheal tube for illuminating the area of tissue of interest;
(b) a camera disposed at the distal end of the endotracheal tube to capture an image of the illuminated tissue;
(c) a first set of conductors extending from said source of light to provide electrical power to said source of light;
(d) a second set of electrical conductors extending from said camera to provide electrical power to said camera and to convey a signal reflective of the image captured by said camera;
(e) a transmitter coupled to said second set of electrical conductors for receiving the signal from said camera and for transmitting the signal;
(f) a receiver for receiving the signal transmitted by the transmitter; and
(g) a display coupled with said receiver for displaying the image represented by the signal.

5. The apparatus as set forth in claim 4 wherein said source of light is a light emitting diode.

6. The apparatus as set forth in claim 4 wherein said source of light is at least one light emitting diode.

7. The apparatus as set forth in claim 4 including a plug and a receptacle for detachably attaching said first and second set of conductors with said transmitter.

8. The apparatus as set forth in claim 4 wherein said display comprises a video monitor.

9. The apparatus as set forth in claim 4 wherein a segment of said first set of conductors is embedded within the wall of the endotracheal tube.

10. The apparatus as set forth in claim 9 including a plug for interconnecting said first set of conductors with a receptacle in said transmitter.

11. The apparatus as set forth in claim 4 wherein a segment of said second set of conductors is embedded within the wall of the endotracheal tube.

12. The apparatus as set forth in claim 11 including a plug for interconnecting said second set of conductors with a receptacle in said transmitter.

13. A method for medical personnel to view in real time an image of tissue at the distal end of an endotracheal tube, said method comprising the steps of:
(a) illuminating the area proximate the distal end of the endotracheal tube with at least one light emitting diode;
(b) providing electrical power to the at least one light emitting diode via a first set of electrical conductors;
(c) imaging the tissue at the distal end of the endotracheal tube with a camera;
(d) further providing electrical power to the camera via a second set of electrical conductors;
(e) conveying a signal reflective of the image captured by the camera via the second set of electrical conductors;
(f) terminating the first and second set of conductors at a transmitter module to provide electrical power to the at least one diode and the camera and to process the signal received from the camera;
(g) transmitting the processed signal;
(h) receiving the processed signal with a receiver; and
(i) displaying an image represented by the signal on a video monitor.

14. The method as set forth in claim 13 including the step of plugging the terminal ends of the first and second set of electrical conductors extending from within the wall of the endotracheal tube into the transmitter module.

15. The method as set forth in claim 13 including the step of plugging the terminal ends of the first and second set of electrical conductors into the transmitter module.
